(19) United States
(12) Patent Application Publication
(10) Pub. No.: US 2026/0251641 A1
(43) Pub. Date: Aug. 27, 2026

(54) OPTICAL NANOSENSORS FOR HYDROLYTIC ENZYME CHARACTERIZATION

(71) Applicant: IOWA STATE UNIVERSITY RESEARCH FOUNDATION, INC., Ames, IA (US)

(72) Inventors: Nigel Forest Reuel, Ames, IA (US); Nathaniel Kallmyer, Whitestown, IN (US)

(21) Appl. No.: 18/831,237

(22) PCT Filed: Apr. 4, 2023
(86) PCT No.: PCT/US2023/065327

§ 371 Date: Oct. 4, 2024

Related U.S. Application Data
(60) Provisional application No. 63/328,170, filed on Apr. 6, 2022.

(51) Int. Cl. G01N 33/542; B82Y 15/00; C12Q 1/34; G01N 33/543

(52) U.S. Cl. CPC G01N 33/542; C12Q 1/34; G01N 33/54346; B82Y 15/00; G01N 2333/916

(57) ABSTRACT

Various embodiments disclosed relate to a sensor assembly probe for determining enzymatic activity. The sensor assembly probe includes an aqueous medium including one or more fluorescent hydrophobic semi-conductive nanoparticles dispersed therein. The assembly further includes an ionic polymer coating at least a portion of a surface of the one or more fluorescent hydrophobic semi-conductive nanoparticles. The assembly further includes a substrate for an enzyme, the substrate disposed between at least two of the one or more fluorescent hydrophobic semi-conductive nanoparticles, the substrate including at least ionic group.

### CROSS-REFERENCE TO RELATED APPLICATION

This application claims the benefit of priority to U.S. Provisional Patent Application Ser. No. 63/328,170 entitled “OPTICAL NANOSENSORS FOR HYDROLYTIC ENZYME CHARACTERIZATION,” filed Apr. 6, 2022, the disclosure of which is incorporated herein in its entirety by reference.

### STATEMENT OF GOVERNMENT SUPPORT

This invention was made with Government support under Grant No. GR-020671-00001 awarded by the USDA. The U.S. Government has certain rights in this invention.

### BACKGROUND

Determining the presence and activity of an enzyme can be useful in many different contexts. In some applications, this can be done indirectly by detecting the presence of byproducts of the reaction between an enzyme and substrate. Indirect detection can be unreliable and potentially expensive. It may, therefore, be desirable to develop improved detection methods and assemblies.

### SUMMARY OF THE DISCLOSURE

Various embodiments disclosed relate to a sensor assembly probe for determining enzymatic activity. The sensor assembly probe includes an aqueous medium including one or more fluorescent hydrophobic semi-conductive nanoparticles dispersed therein. The assembly further includes an ionic polymer coating at least a portion of a surface of the one or more fluorescent hydrophobic semi-conductive nanoparticles. The assembly further includes a substrate for an enzyme, the substrate disposed between at least two of the one or more fluorescent hydrophobic semi-conductive nanoparticles, the substrate including at least ionic group.

There are many advantages associated with the assemblies and methods disclosed herein, some of which are unexpected. For example, according to various embodiments, the assembly can be easily and rapidly synthesized and detect the presence of an enzyme and depletion of an enzyme substrate or analogue thereof in real time. Additionally, according to various embodiments, even though the substrate can be fluorogenic or colorimetric, it is not necessary and therefore a natural substrate analogue can be used. According to various embodiments, the assembly is versatile, for example, it can screen different types of substrate and enzyme combinations. According to various embodiments, depletion of substrate can be correlated with signal change to predict quantitative rate constants. According to various further embodiments, comparison with an established colorimetric assay can demonstrate high performance in complex, otherwise-difficult samples. According to various further embodiments, the assemblies and methods disclosed herein can be used to rapidly track changes in enzyme activity to monitor damage or to perform optimization operations.

According to further embodiments, the assembly's versatility, compact size scale, and penetrating near infrared spectral window can make it suitable for a range of enzyme-related applications. According to further embodiments, the assemblies and methods disclosed herein may be suitable, for example, in industrial biotechnology by offering speed and simplicity enhancement to routine optimization studies where new mutant hydrolase enzymes are screened to determine optimal operating conditions. According to various embodiments, for example in medical applications, the methods and assemblies may be used for in vivo zymography, or the tracking of temporal and spatial dynamics of health-related enzymes such as MMPs from tumors. According to various embodiments, for example, in drug development, the assemblies and methods may be used in the design and screening of hydrolase inhibitors. According to various embodiments, for example, in food production, the assemblies and methods may be used to test and design cost-effective immobilization strategies for hydrolases in routine processing such as saccharification. According to various embodiments, the assemblies and methods can be modified to increase portability, sensitivity, and to reduce cost. Furthermore, according to various embodiments, sensor stability, sensitivity, and selectivity can be further optimized by modifying sensor manufacturing conditions.

### BRIEF DESCRIPTION OF THE FIGURES

The drawings illustrate generally, by way of example, but not by way of limitation, various embodiments discussed in the present document.

FIGS. 1A-1C are schematic diagrams showing the operation of a sensor probe.

FIGS. 2A-2D are graphs showing results obtained using the sensor probe of FIGS. 1A-1B.

### DETAILED DESCRIPTION

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section.

In the methods described herein, the acts can be carried out in any order without departing from the principles of the disclosure, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range, and includes the exact stated value or range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%.

The term “weight-average molecular weight” as used herein refers to M_{w}, which is equal to ΣMᵢ²nᵢ/ΣMᵢnᵢ, where nᵢ is the number of molecules of molecular weight Mᵢ. In various examples, the weight-average molecular weight can be determined using light scattering, small angle neutron scattering, X-ray scattering, and sedimentation velocity.

This disclosure is directed towards various embodiments of a sensor assembly probe. The sensor assembly probe can be used for determining enzymatic activity. The sensor assembly can include one or more fluorescent semi-conductive nanoparticles disposed in an aqueous medium. The sensor assembly probe further includes a coating. The coating coats at least a portion of a surface of the fluorescent semi-conductive nanoparticle. The assembly further includes a substrate for a predetermined enzyme. The coating and the substrate are each ionic species. That is, the substrate is cationic or anionic and the coating is cationic or anionic. For example, the substrate can be anionic and the coating can be cationic or the substrate can be cationic and the coating can be anionic.

In operation, the nanoparticles have a detectible fluorescent emission when dispersed in the aqueous medium. The substrate is positioned between at least two single-wall nanotubes. The substrate and the coating on the single-wall nanotubes adhere through electrostatic interactions. Upon contact with the predetermined enzyme, the substrate can be hydrolyzed or otherwise cleaved. Without the substrate being present to prevent agglomeration of the singe-wall nanotubes, the nanotubes agglomerate. This changes the dielectric environment of the nanoparticle and thus changes the fluorescent signal of the nanoparticle. For example the fluorescent signal can decrease or even disappear. The change in fluorescent signal indicates that the predetermined enzyme of interest is present in solution.

The nanoparticles can include any suitable material. Examples of suitable materials can include a ceramic material (e.g., aluminum oxide or copper(II) oxide), a polymer, a glass-ceramic, a composite, a metal carbide (e.g., SiC), a nitride (e.g., aluminum nitride, silicon nitride), a metal (e.g., Al, Cu, Au, Ag), a non-metal (e.g., graphite and carbon). The nanoparticle can have any suitable morphology. For example, the morphology of the nanoparticle can be chosen from a nanosphere, a nanorod, a nanofiber, a nanotube, a nanostar, a nanocup, or combinations thereof. At least one of a length, width, and diameter of the nanoparticle is in a range of from about 0.5 nm to about 10,000 nm, about 1 nm to about 100 nm, about 10 nm to about 50 nm, about 100 nm to about 2,500 nm, about 2,500 nm to about 10,000 nm, or less than, equal to, or greater than about 0.5 nm, 0.7, 1, 25, 50, 100, 500, 1,000, 1,500, 2,000, 2,500, 3,000, 3,500, 4,000, 4,500, 5,000, 5,500, 6,000, 6,500, 7,000, 7,500, 8,000, 8,500, 9,000, 9,500, or about 10,000 nm. Generally, nanoparticles in which at least one of a length, width, and diameter of the nanoparticle is in a range of from about 0.5 nm to about 100 nm are classified as ultrafine nanoparticles. Generally, nanoparticles in which at least one of a length, width, and diameter of the nanoparticle is in a range of from about 100 nm to about 2,500 nm are classified as fine nanoparticles. Generally, nanoparticles in which at least one of a length, width, and diameter of the nanoparticle is in a range of from about 2,500 nm to about 10,000 nm are classified as coarse nanoparticles. The morphology of the nanoparticles can be uniform.

The sensor assembly probe can include a plurality of the nanoparticles. Respective individual nanoparticles can have at least one of substantially the same morphology, substantially the same dimensions, and have substantially the same composition. Alternatively, the respective individual nanoparticles can differ in at least one of their morphologies, dimensions, and compositions. The plurality of nanoparticles can be heterogeneously or homogenously distributed in the aqueous medium.

When at least one nanoparticle is dispersed in the aqueous medium (e.g., free of agglomeration with another nanoparticle) the nanoparticle fluoresces. In some embodiments, the nanoparticle can fluoresce at wavelengths ranging from about 800 nm to about 1500 nm, 950 nm to about 1100 nm, or less than, equal to, or greater than about 800, 810, 820, 830, 840, 850, 860, 870, 880, 890, 900, 910, 920, 930, 940, 950, 960, 970, 980, 990, 1000, 1100, 1200, 1300, 1400, or about 1500 nm. In embodiments where the sensor assembly probe includes a plurality of nanoparticles, respective nanoparticles can fluoresce at substantially the same frequency.

In other embodiments where the sensor assembly includes a plurality of nanoparticles, respective nanoparticles can fluoresce at different frequencies. For example, the respective fluorescent signals emitted by the first fluorescent semi-conductive nanoparticle and the second fluorescent semi-conductive nanoparticle have intensities of fluorescence that differ by about 0 to 100%, about 0 to 20% or less than, equal to, or greater than about 0%, 5, 10, 15, 20, 25, 30, 35, 40, 45, 50, 55, 60, 65, 70, 75, 80, 85, 90, 95, or 100%.

The substrate for the predetermined enzyme can be selected from many suitable candidates. In some embodiments, the substrate can be a protein, a peptide, a small organic molecule, an organic macromolecule or a mixture thereof. An example of a suitable substrate is phytate. Examples of suitable substrates include those with a bond that is hydrolyzable by the predetermined enzyme. Examples of such bonds can include a phosphoester bond, an ester bond, a glycosylic bond, an ether bond, a peptide bond, an acid anhydride bond, a halide bond, a phosphorous-sulfur bond, a sulfur-sulfur bond, a carbon-phosphorous bond, a carbon-sulphur bond, or a combination thereof.

The sensor assembly probe can include a plurality of substrates. The respective protein, peptide, small organic molecule, organic macromolecule or a mixture thereof can differ by composition, weight-average-molecular weight or a combination thereof. The respective protein, peptide, small organic molecule, organic macromolecule or a mixture thereof can further be substrates of the same enzyme or different enzymes.

The extent to which coating coats the nanoparticle can be tuned to any suitable degree. For example, the coating can coat from about 20% to about 100% of surface area of the nanoparticle, about 50% surface area to about 90 % surface area, about 90% surface area to about 100% surface area, or less than, equal to, or greater than about 20%, 25, 30, 35, 40, 45, 50, 55, 60, 65, 70, 75, 80, 85, 90, 95, or 100% surface area. The coating can include a protein, a peptide, a small organic molecule, an organic macromolecule, an organic acid, or a mixture thereof. In some examples the coating can include a polysaccharide. An example of a suitable polysaccharide can include chitosan.

The predetermined enzyme in the sensor probe assembly can be any suitable enzyme. The enzyme is selected to react with the substrate. The sensor assembly probe can include more than one types of enzyme. In embodiments that include more than one enzyme, the enzymes can be the same enzyme or a mixture of different enzymes. Where different enzymes are present in the assembly, the different enzymes can be adapted to react with different substrates. The substrate that a particular enzyme reacts with may be present in the assembly or may not be present in the assembly.

The predetermined enzyme or enzymes may belong to any class of enzymes. For example, the enzyme or enzymes may be classified as a hydrolase (alternatively known as an EC 3 enzyme). The hydrolase can be classified by the bond it acts upon. For example, the hydrolase can be chosen from a phytase, an esterase, nuclease, phosphodiesterase, lipase, phosphatase, DNA glycosylase, glycoside hydrolase, proteases, peptidase, acid anhydride hydrolase, helicase, GTPase, or mixtures thereof.

The sensor assembly can be used according to any suitable method. According to various embodiments, the method can include dispensing the nanoparticles that are at least partially coated with the coating. When the nanoparticles are solubilized, they produce a fluorescent emission. The initial fluorescence is measured. In some embodiments where the assembly includes a mixture of nanoparticles that produce different fluorescent emissions, multiple emissions may be measured.

The enzyme or mixture of enzymes are then contacted with the nanoparticles and substrates. If an enzyme is associated with a particular substrate, the substrate will be reacted and bind to the nanoparticle and the fluorescent emission of the nanoparticle to which the substrate is attached will be changed. Thus, a measured second fluorescent emission will have a different intensity than the first fluorescent emission or there will be no fluorescent emission.

Measuring a second fluorescent emission that is different than the first fluorescent emission confirms the presence of a predetermined enzyme. In embodiments where nanoparticles having different fluorescent emissions and different substrates attached thereto are present, a decrease in the emission in one or both of the nanoparticles can indicate the presence of two different predetermined enzymes. In this manner, the presence of one or more enzymes in a mixture of enzymes or another constituent of a solution can be confirmed.

Additionally, the rate of reaction between the predetermined enzyme and a substrate can be determined by monitoring the rate at which the fluorescent emission intensity changes.

The sensor assembly can be formed according to any suitable method. For example, the assembly can be formed by dispersing the nanoparticles in an aqueous medium. The nanoparticles can then be mixed with the coating. Mixing can be accomplished for example through sonication.

### EXAMPLES

Various embodiments of the present disclosure can be better understood by reference to the following Examples which are offered by way of illustration. The present disclosure is not limited to the Examples given herein.

### Methods

### Chitosan Gel Qualitative Characterization

Chitosan solution was prepared by dissolving 10 mg/mL chitosan (Sigma 448877) in 1% m/v glacial acetic acid in deionized water. This solution was then degassed under vacuum. SWNT (Chasm SG65i) were added to 2 mL of this solution at a ratio of 0.5 mg SWNT per 1 mL solution. This mixture was then sonicated (Qsonica Q125, ⅛″) at 5 W for 40 min. The resulting suspension was centrifuged at 10,000×g for 5 min, and chitosanSWNT were collected as supernatant. Phytate solution (pH=7) was prepared by neutralizing 50% w/v phytic acid with sodium hydroxide. Two chitosan-phytate aggregate samples were prepared by adding 100 μL phytate solution to 5 mL samples of chitosan solution inside centrifuge tubes. Following aggregation, 100 μL of 10 mg/mL phytase solution was added to one sample, and 100 μL of deionized water was added to the other. After 20 minutes, aggregates were removed from the centrifuge tubes and observed outside of liquid. Two chitosan-SWNT-phytate aggregate samples were similarly prepared by adding 1 μL phytate solution to a 1 mL combination of 1:1 chitosan-SWNT: chitosan solution. After aggregation, 10 μL of 10 mg/mL phytase solution was added to one sample, and 10 μL deionized water added to the other.

### Chitosan-SWNT Phytase Assay

Chitosan and sensor solutions were prepared as described above. The chitosan-SWNT were then diluted fivefold in the previously prepared chitosan solution. To assay phytase activity, 80 μL of the diluted chitosan-SWNT were added to wells of a 96-well plate. 80 μL of 1% phytate solution (pH=7) were then added to each well. The wells were then incubated at room temperature for 40 min. Assays were performed on a custom-built fluorescent nIR wellplate scanner 10. Activity assays were performed by, first, starting a 20-minute fluorescent scan. 20 μL wheat phytase samples were added to each of these wells.

### Results and Discussion

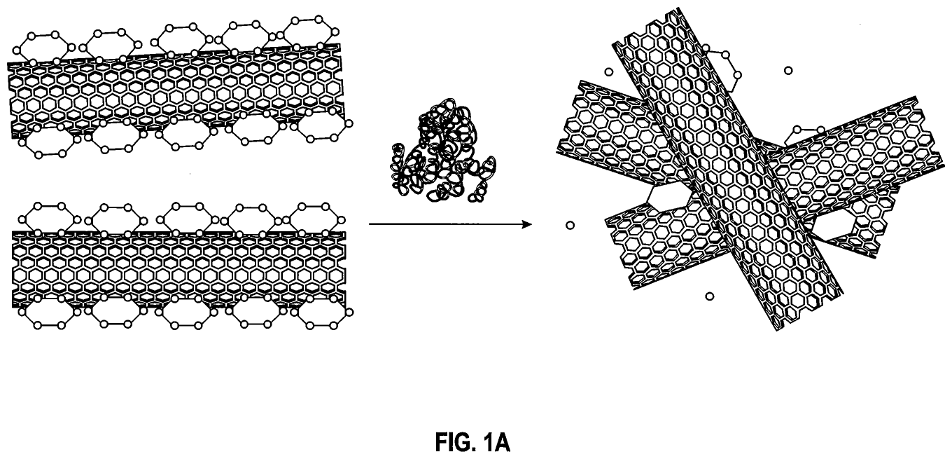

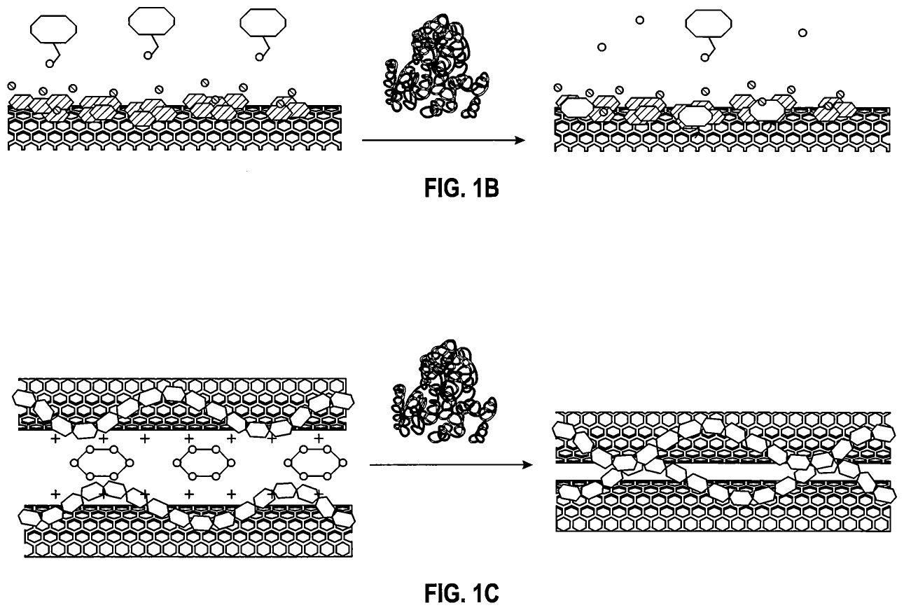

Several different functionalization strategies attempted, as shown in FIG. 1. Sensors were initially prepared by sonicating SWNT directly in 50% phytate solution, a process demonstrated effective for dispersion of oxidized multi-walled carbon nanotubes (FIG. 1A). While this suspension method was successful with semiconducting SWNT in an isolated case, this method was not reproducible. Further study with attention to SWNT defects may elucidate the cause of this inconsistency. This could be performed with a more controlled method of dispersion, such as lower energy shear mixing. The second approach utilized a phosphorylated substrate analog, riboflavin phosphate, and lignosulfonic acid-SWNT to report phosphatase activity. Riboflavin phosphate was selected as a substrate for its phosphoester bond and well-documented quenching of SWNT sensors. Upon degradation, riboflavin would enter the hydrophobic corona phase and cause quenching (FIG. 1B). This approach was effective with purified enzyme samples but suffered from confounding effects when tested against crude feed samples. Use of a substrate analog rather than phytase further confounded testing, as responses likely correlated with enzyme selectivity towards the analog rather than activity towards phytate.

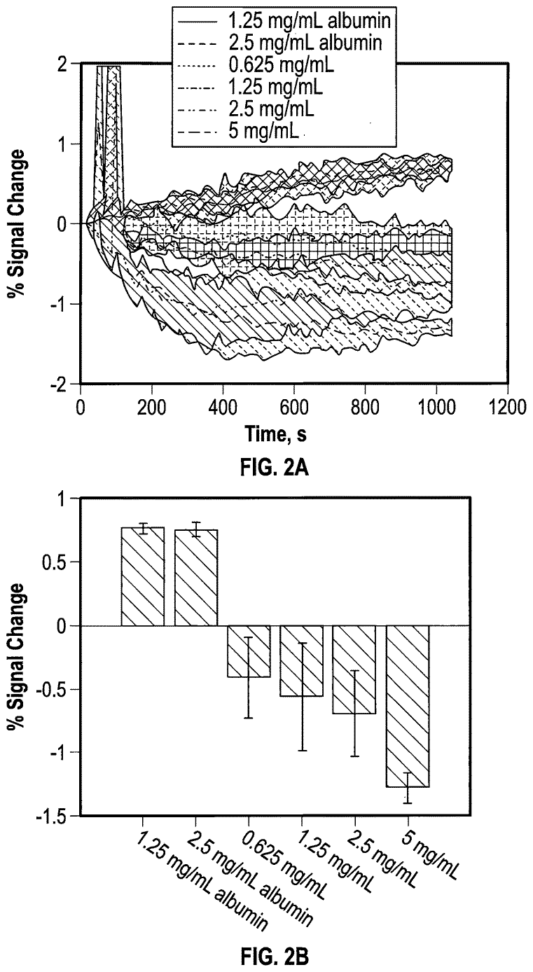

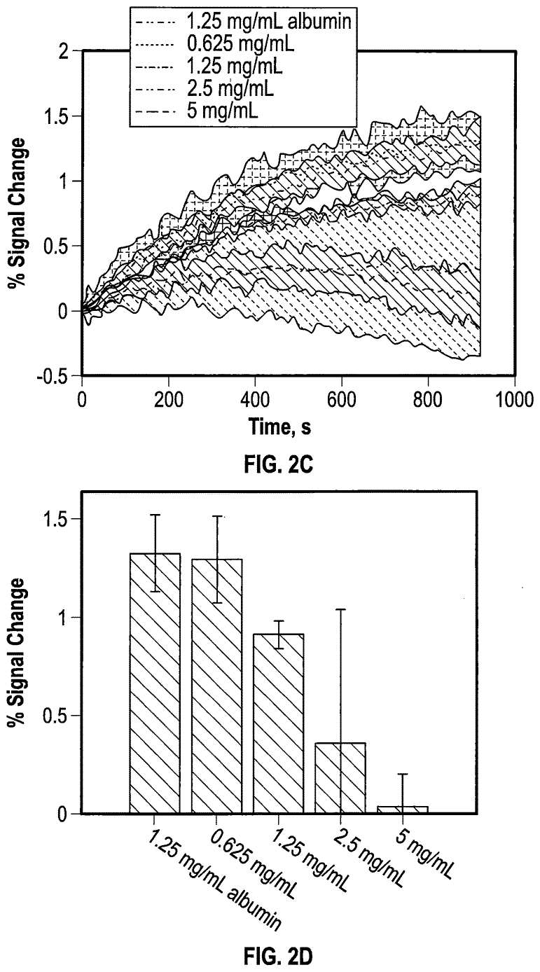

FIGS. 2A-2D show results obtained using the probe assembly described herein with respect to FIG. 1C. FIG. 2A shows response traces to albumin controls and different concentrations of phytate. FIG. 2B shows normalized end-signals from FIG. 2A. FIG. 2C shows response traces from replicate experiment. Response trace begins immediately after addition of phytase to mitigate shift caused by jostling of detector. FIG. 2D shows end-signals from FIG. 2C. Shaded regions and error bars in FIGS. 2A and 2C indicate a single standard deviation (n=4).

### Additional Embodiments

The following exemplary embodiments are provided, the numbering of which is not to be construed as designating levels of importance:

Aspect 1 provides a sensor assembly probe for determining enzymatic activity comprising:
an aqueous medium comprising one or more fluorescent hydrophobic semi-conductive nanoparticles dispersed therein;
an ionic polymer coating at least a portion of a surface of the one or more fluorescent hydrophobic semi-conductive nanoparticles;
a substrate for an enzyme, the substrate disposed between at least two of the one or more fluorescent hydrophobic semi-conductive nanoparticles, the substrate comprising at least ionic group.

Aspect 2 provides the sensor assembly probe of Aspect 1 wherein the ionic polymer at least a portion of the surface of the one or more fluorescent hydrophobic semi-conductive nanoparticles comprises a cationic polymer.

Aspect 3 provides the sensor assembly probe of Aspect 1 wherein the ionic polymer at least a portion of the surface of the one or more fluorescent hydrophobic semi-conductive nanoparticles comprises an ionic polymer.

Aspect 4 provides the sensor assembly probe of any one of Aspects 1-3, wherein the ionic group of the substrate comprises an anionic group.

Aspect 5 provides the sensor assembly probe of any one of Aspects 1-3, wherein the ionic group of the substrate comprises a cationic group.

Aspect 6 provides the sensor assembly probe of any one of Aspects 1-5, wherein a morphology of the fluorescent hydrophobic semi-conductive nanoparticle comprises a nanosphere, a nanorod, a nanofiber, a nanotube, a nanostar, a nanocup, or combinations thereof.

Aspect 7 provides the sensor assembly probe of any one of Aspects 1-6, wherein at least one of a length, width, and diameter of the one or more fluorescent hydrophobic semi-conductive nanoparticles is in a range of from about 0.5 nm to about 100 nm.

Aspect 8 provides the sensor assembly probe of any one of Aspects 1-7, wherein a particle size of the one or more fluorescent hydrophobic semi-conductive nanoparticles is in a range of from about 10 nm to about 50 nm.

Aspect 9 provides the sensor assembly probe of any one of Aspects 1-8, wherein the one or more fluorescent hydrophobic semi-conductive nanoparticles comprises a ceramic, a polymer, a metal carbide, a nitride, a metal, graphite, carbon, or a mixture thereof.

Aspect 10 provides the sensor assembly probe of any one of Aspects 1-9, wherein the one or more fluorescent hydrophobic semi-conductive nanoparticles comprise a carbon nanotube.

Aspect 11 provides the sensor assembly probe of any one of Aspects 1-10, wherein the one or more fluorescent hydrophobic semi-conductive nanoparticles independently fluoresce at frequency ranging from about 800 nm to about 1500 nm.

Aspect 12 provides the sensor assembly probe of any one of Aspects 1-11, wherein one or more fluorescent hydrophobic semi-conductive nanoparticles independently fluoresce at frequency ranging from about 950 nm to about 1100 nm

Aspect 13 provides the sensor assembly probe of any one of Aspects 1-12, wherein the substrate is a protein, a peptide, or a mixture thereof.

Aspect 14 provides the sensor assembly probe of any one of Aspects 1-13, wherein the substrate comprises phytate, bovine serum albumin, citrus pectin, carboxymethyl cellulose, or a mixture thereof.

Aspect 15 provides the sensor assembly probe of any one of Aspects 1-14, wherein the substrate comprises a bond that is hydrolyzable by the predetermined enzyme.

Aspect 16 provides the sensor assembly probe of any one of Aspects 1-15, wherein the substrate comprises a phosphoester bond, an ester bond, a glycosylic bond, an ether bond, a peptide bond, an acid anhydride bond, a halide bond, a phosphorous-sulfur bond, a sulfur-sulfur bond, a carbon-phosphorous bond, a carbon-sulfur bond, or a mixture thereof.

Aspect 17 provides the sensor assembly probe of any one of Aspects 1-16, wherein the ionic polymer coats about 20% to about 100% of surface area of the one or more fluorescent hydrophobic semi-conductive nanoparticles.

Aspect 18 provides the sensor assembly probe of any one of Aspects 1-17, wherein the ionic polymer coats about 90% to about 100% of surface area of the one or more fluorescent hydrophobic semi-conductive nanoparticles.

Aspect 19 provides the sensor assembly probe of any one of Aspects 1-18, wherein the ionic polymer coats about 95% to about 100% of surface area of the one or more fluorescent hydrophobic semi-conductive nanoparticles.

Aspect 20 provides the sensor assembly probe of any one of Aspects 1-19, wherein the ionic polymer comprises a polysaccharide.

Aspect 21 provides the sensor assembly probe of any one of Aspects 1-20, wherein the ionic polymer comprises chitosan.

Aspect 22 provides the sensor assembly probe of any one of Aspects 1-21, wherein the substrate is the substrate for the predetermined enzyme.

Aspect 23 provides the sensor assembly probe of any one of Aspects 1-22, wherein the substrate comprises phytate.

Aspect 24 provides the sensor assembly probe of any one of Aspects 1-23, wherein the predetermined enzyme is a hydrolase.

Aspect 25 provides the sensor assembly probe of Aspect 24, wherein the hydrolase comprises a phytase, an esterase, a nuclease, a phosphodiesterase, a lipase, a phosphatase, a DNA glycosylase, a glycoside hydrolase, a protease, a peptidase, an acid anhydride hydrolase, a helicase, a GTPase, or a mixture thereof.

Aspect 26 provides the sensor assembly probe of any one of Aspects 1-25, wherein at least one of the one or more the fluorescent hydrophobic semi-conductive nanoparticles is a first fluorescent hydrophobic semi-conductive nanoparticle and the assembly further comprises a second fluorescent hydrophobic semi-conductive nanoparticle.

Aspect 27 provides the sensor assembly probe of Aspect 26, wherein the first fluorescent hydrophobic semi-conductive nanoparticle and the second fluorescent hydrophobic semi-conductive nanoparticle have substantially the same composition.

Aspect 28 provides the sensor assembly probe of Aspect 27, wherein the first fluorescent hydrophobic semi-conductive nanoparticle and the second fluorescent hydrophobic semi-conductive nanoparticle have different compositions.

Aspect 29 provides the sensor assembly probe of any one of Aspects 26-28, wherein the first fluorescent hydrophobic semi-conductive nanoparticle and the second fluorescent hydrophobic semi-conductive nanoparticle fluoresce at different frequencies.

Aspect 30 provides the sensor assembly probe of Aspect 29, wherein the respective fluorescent signals emitted by the first fluorescent hydrophobic semi-conductive nanoparticle and the second fluorescent hydrophobic semi-conductive nanoparticle have frequencies of fluorescence that differ by about 0% to about 100%, relative to each other.

Aspect 31 provides the sensor assembly probe of any one of Aspects 29 or 30, wherein the respective fluorescent signals emitted by the first fluorescent hydrophobic semi-conductive nanoparticle and the second fluorescent hydrophobic semi-conductive nanoparticle have frequencies of fluorescence that differ by about 0% to about 20%, relative to each other.

Aspect 32 provides the sensor assembly probe of any one of Aspects 26-31, wherein the first fluorescent hydrophobic semi-conductive nanoparticle and the second fluorescent hydrophobic semi-conductive nanoparticle are homogenously dispersed in the aqueous medium.

Aspect 33 provides the sensor assembly probe of any one of Aspects 26-32, wherein the first fluorescent hydrophobic semi-conductive nanoparticle and the second fluorescent hydrophobic semi-conductive nanoparticle are heterogeneously distributed in the aqueous medium.

Aspect 34 provides the sensor assembly probe of any one of Aspects 1-33, wherein the substrate is a first substrate and the assembly further comprises a second substrate.

Aspect 35 provides the sensor assembly probe of Aspect 34, wherein the first substrate and the second substrate comprise different substrates for different enzymes.

Aspect 36 provides a sensor assembly comprising the probe of any one of Aspects 1-35, the sensor assembly further comprising the predetermined enzyme dispersed about the aqueous medium.

Aspect 37 provides the sensor assembly of Aspect 36, wherein the predetermined enzyme is a first enzyme and the assembly further comprises a second enzyme.

Aspect 38 provides the sensor assembly probe of any one of Aspects 1-37, or the sensor assembly of any one of Aspects 36 or 37, wherein
the hydrophobic semi-conductive nanoparticle comprises a carbon nanotube;
the ionic polymer comprises chitosan;
the substrate comprises a phytate; and
the predetermined enzyme is phytase.

Aspect 39 provides a method of using the sensor assembly probe of any one of Aspects 1-38, the method comprising:
measuring a first fluorescent frequency emission of the probe;
contacting the probe and the predetermined enzyme; and
measuring a second fluorescent frequency emission of the probe, wherein the second fluorescent frequency emission is less than the first fluorescent frequency emission and indicates that at least a portion the substrate has reacted with the predetermined enzyme.

Aspect 40 provides the method of Aspect 39, wherein the second fluorescent frequency emission is zero.

Aspect 41 provides the method of any one of Aspects 39 or 40, wherein a mixture of enzymes comprises the predetermined enzyme.

Aspect 42 provides the method of any one of Aspects 39-41, further comprising determining a rate of reaction between the substrate and the predetermined enzyme.

Aspect 43 provides the method of Aspect 42, wherein determining a rate of reaction comprises measuring a plurality of fluorescent signals over a predetermined amount of time to quantify the amount of substrate that is consumed by the predetermined enzyme over the predetermined amount of time.

Aspect 44 provides the method of any one of Aspects 39-43, wherein at least two of the one or more fluorescent hydrophobic semi-conductive nanoparticles at least partially adhere to each other after the predetermined enzyme reacts with the substrate.

Aspect 45 provides a method of making the sensor assembly probe of any one of Aspects 1-44, the method comprising:
dispersing the one or more fluorescent hydrophobic semi-conductive nanoparticles and the substrate in an aqueous medium; and
mixing the fluorescent hydrophobic semi-conductive nanoparticle and the substrate, to form the sensor assembly.

Aspect 46 provides the method of Aspect 45, wherein mixing comprises sonication.

Aspect 47 provides the method of any one of Aspects 45 or 46, further comprising contacting the probe and the predetermined enzyme.

1. A sensor assembly probe for determining enzymatic activity comprising:
an aqueous medium comprising one or more fluorescent hydrophobic semi-conductive nanoparticles dispersed therein;
an ionic polymer coating at least a portion of a surface of the one or more fluorescent hydrophobic semi-conductive nanoparticles;
a substrate for an enzyme, the substrate disposed between at least two of the one or more fluorescent hydrophobic semi-conductive nanoparticles, the substrate comprising at least ionic group.

2. The sensor assembly probe of claim 1, wherein the ionic polymer at least a portion of the surface of the one or more fluorescent hydrophobic semi-conductive nanoparticles comprises a cationic polymer.

3. The sensor assembly probe of claim 1, wherein a morphology of the fluorescent hydrophobic semi-conductive nanoparticle comprises a nanosphere, a nanorod, a nanofiber, a nanotube, a nanostar, a nanocup, or combinations thereof.

4. The sensor assembly probe of claim 1, wherein at least one of a length, width, and diameter of the one or more fluorescent hydrophobic semi-conductive nanoparticles is in a range of from about 0.5 nm to about 100 nm.

5. The sensor assembly probe of claim 1, wherein the one or more fluorescent hydrophobic semi-conductive nanoparticles comprise a carbon nanotube.

6. The sensor assembly probe of claim 1, wherein the one or more fluorescent hydrophobic semi-conductive nanoparticles independently fluoresce at frequency ranging from about 800 nm to about 1500 nm.

7. The sensor assembly probe of claim 1, wherein the substrate is a protein, a peptide, or a mixture thereof.

8. The sensor assembly probe of claim 1, wherein the substrate comprises a bond that is hydrolyzable by the predetermined enzyme.

9. The sensor assembly probe of claim 1, wherein the ionic polymer coats about 90% to about 100% of surface area of the one or more fluorescent hydrophobic semi-conductive nanoparticles.

10. The sensor assembly probe of claim 1, wherein the ionic polymer comprises chitosan.

11. The sensor assembly probe of claim 1, wherein the substrate is the substrate for the predetermined enzyme.

12. The sensor assembly probe of claim 1, wherein the predetermined enzyme is a hydrolase.

13. The sensor assembly probe of claim 12, wherein the hydrolase comprises a phytase, an esterase, a nuclease, a phosphodiesterase, a lipase, a phosphatase, a DNA glycosylase, a glycoside hydrolase, a protease, a peptidase, an acid anhydride hydrolase, a helicase, a GTPase, or a mixture thereof.

14. The sensor assembly probe of claim 1, wherein at least one of the one or more the fluorescent hydrophobic semi-conductive nanoparticles is a first fluorescent hydrophobic semi-conductive nanoparticle and the assembly further comprises a second fluorescent hydrophobic semi-conductive nanoparticle.

15. The sensor assembly probe of claim 1, wherein the first fluorescent hydrophobic semi-conductive nanoparticle and the second fluorescent hydrophobic semi-conductive nanoparticle fluoresce at different frequencies.

16. A sensor assembly comprising the probe of claim 1, the sensor assembly further comprising the predetermined enzyme dispersed about the aqueous medium.

17. (canceled)

18. (canceled)

19. A method of using the sensor assembly probe of claim 1, the method comprising:
measuring a first fluorescent frequency emission of the probe;
contacting the probe and the predetermined enzyme; and
measuring a second fluorescent frequency emission of the probe, wherein the second fluorescent frequency emission is less than the first fluorescent frequency emission and indicates that at least a portion the substrate has reacted with the predetermined enzyme.

20. (canceled)

21. A method of making the sensor assembly probe of claim 1, the method comprising:
dispersing the one or more fluorescent hydrophobic semi-conductive nanoparticles and the substrate in an aqueous medium; and
mixing the fluorescent hydrophobic semi-conductive nanoparticle and the substrate, to form the sensor assembly.

22. The method of claim 21, wherein mixing comprises sonication.

23. The method of claim 21, further comprising contacting the probe and the predetermined enzyme.
